(19) United States
(12) Patent Application Publication
(10) Pub. No.: US 2022/0165381 A1
(43) Pub. Date: May 26, 2022

(54) SYSTEMS AND METHODS FOR DETECTING COMPLIANCE WITH A MEDICATION REGIMEN

(71) Applicant: Electronic Caregiver, Inc., Las Cruces, NM (US)

(72) Inventors: Anthony Dohrmann, Las Cruces, NM (US); Jeremy Keys, Las Cruces, NM (US)

(21) Appl. No.: 17/103,677

(22) Filed: Nov. 24, 2020

(51) Int. Cl. G16H 20/10; H04N 7/15; G06K 9/00; G10L 15/26

(52) U.S. Cl. CPC G16H 20/10; H04N 7/157; G10L 15/26; G06K 9/00375; G06K 9/00228; G06K 9/00355

(57) ABSTRACT

Exemplary embodiments include a computing device configured to dynamically display a specific, structured interactive animated conversational graphical user interface paired with a prescribed functionality directly related to the interactive graphical user interface's structure. Also included is a first computer vision model and a second computer vision model. The first computer vision model is configured to track a hand of a human being, and the second computer vision model is configured to track a face of a human being. The computing device is programed with heuristic logic. The heuristic logic infers if (i) the hand is visible, (ii) the face is visible, (iii) the back of the hand is visible, and (iv) the face is occluded, then a medication has been taken by a human.

### FIELD OF THE TECHNOLOGY

Embodiments of the disclosure relate to computing devices programmed to detect compliance with a medication regimen.

### SUMMARY

Exemplary embodiments include a computing device configured to dynamically display a specific, structured interactive animated conversational graphical user interface paired with a prescribed functionality directly related to the interactive graphical user interface's structure. Also included is a first computer vision model and a second computer vision model. The first computer vision model is configured to track a hand of a human, and the second computer vision model is configured to track a face of a human. The computing device is programed with heuristic logic. The heuristic logic infers if (i) the hand is visible, (ii) the face is visible, (iii) the back of the hand is visible, and (iv) the face is occluded, then a medication has been taken by the human being.

Further exemplary embodiments include a computer vision model configured to track a throat of the human to detect a swallow by the human. Also a computer vision model may be configured to detect a pill type. The computing device of claim may be any form of a computing device, including a personal computer, laptop, tablet, or mobile device. Additionally, upon initiation, a user is provided one or more options to select a desired method for data entry, including voice, type, touch or combinations thereof without having to switch back and forth. The user provided data is validated based on characteristics defined within the specific, structured interactive animated conversational graphical interface. The user provided data may be further validated against external data stored in a cloud-based database.

The specific, structured interactive animated conversational graphical user interface according to many embodiments may complete and update a database entry. The specific, structured interactive animated conversational graphical user interface may convert text data to voice data for storage and for use in human conversation. It may also convert response data to audio files using cloud-based text-to-speech solutions capable of being integrated into a web browser based avatar in the form of a human.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

FIG. 1 shows an exemplary depth camera.

FIG. 2 is a flow chart of an exemplary method for detecting compliance with a medication regimen.

FIG. 3 shows an exemplary specific, structured interactive animated conversational graphical user interface with an avatar in the form of a human.

FIG. 4 shows another exemplary specific, structured interactive animated conversational graphical user interface with an avatar in the form of a human.

### DETAILED DESCRIPTION

In the following description, for purposes of explanation, numerous specific details are set forth in order to provide a thorough understanding of the disclosure. It will be apparent, however, to one skilled in the art, that the disclosure may be practiced without these specific details. In other instances, structures and devices may be shown in block diagram form only in order to avoid obscuring the disclosure.

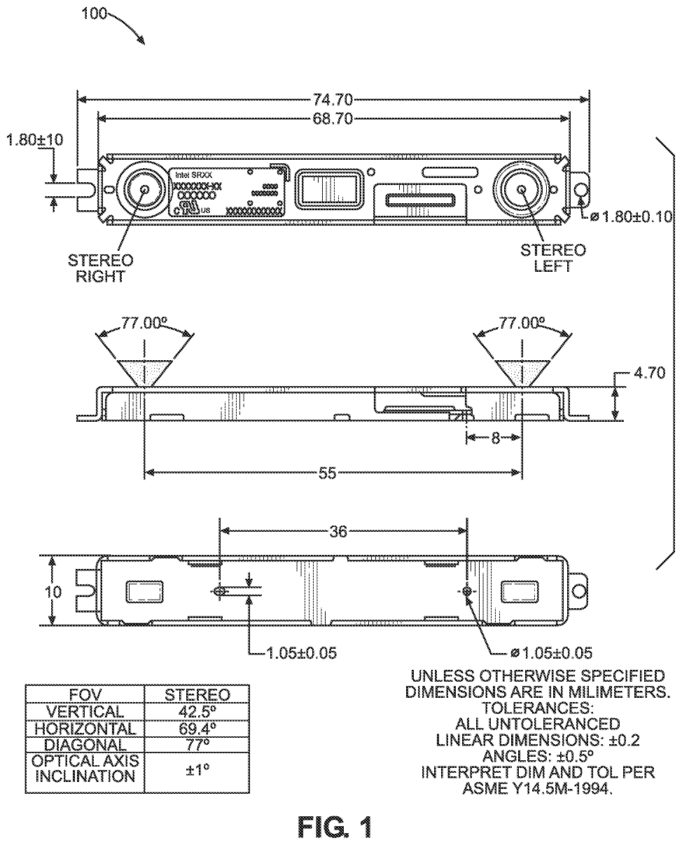

FIG. 1 shows an exemplary depth camera 100 as claimed herein. For example, the Intel®RealSense™ D400 series is a stereo vision depth camera system. The subsystem assembly contains stereo depth module and vision processor with USB2.0/USB 3.1 Gen1 or MIPI1 connection to the host processor. The small size and ease of integration of the camera sub system provides system integrators flexibility to design into a wide range of products. Thelntel®RealSenseTMD400 series also offers complete depth cameras integrating vision processor, stereo depth module, RGB sensor with color image signal processing and Inertial Measurement Unit2 (IMU). The depth cameras are designed for easy setup and portability making them ideal for makers, educators, hardware prototypes and software development. The Intel®RealSense™D400 series is supported with cross-platform and open source Intel®RealSense™ SDK 2.0.

The Intel®RealSense™ D400 series depth camera uses stereo vision to calculate depth. The stereo vision implementation consists of a left imager, right imager, and an optional infrared projector. The infrared projector projects non-visible static IR pattern to improve depth accuracy in scenes with low texture. The left and right imagers capture the scene and sends imager data to the depth imaging (vision) processor, which calculates depth values for each pixel in the image by correlating points on the left image to the right image and via shift between a point on the left image and the right image. The depth pixel values are processed to generate a depth frame. Subsequent depth frames create a depth video stream. According to exemplary embodiments, these depth frames are analyzed as described and claimed herein.

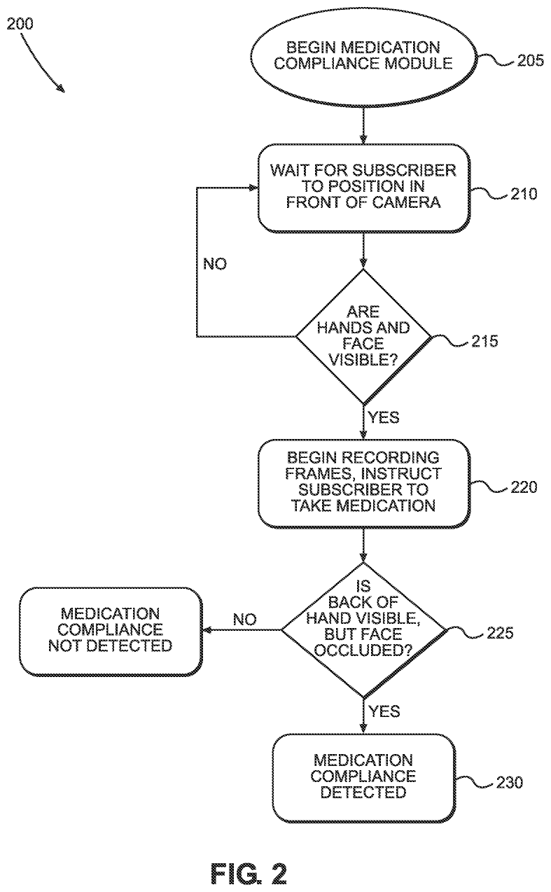

FIG. 2 is a flow chart of an exemplary method 200 for detecting compliance with a medication regimen.

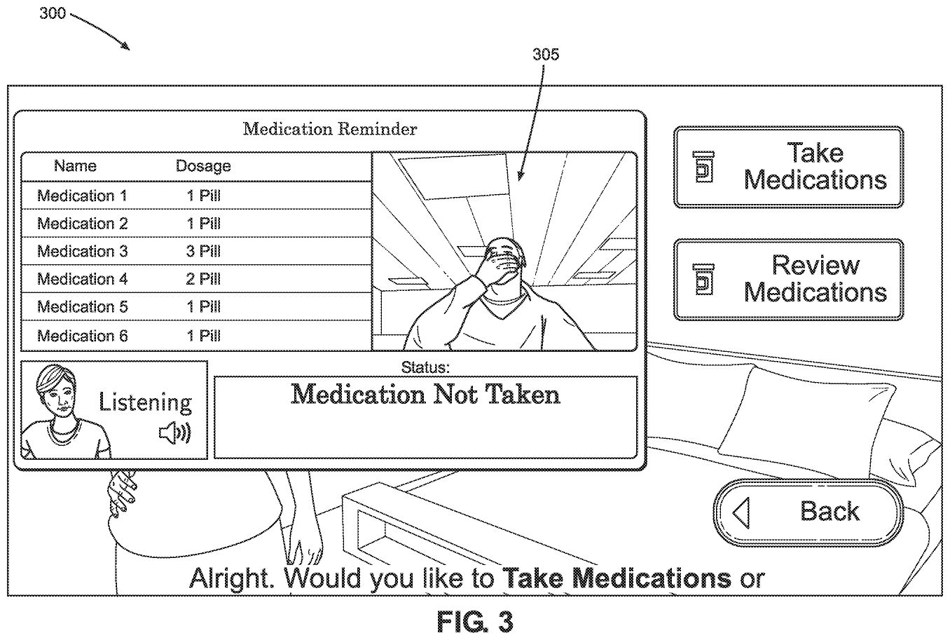

At step 205, a medication compliance module is launched. For example, upon launching a user may be shown the exemplary specific, structured interactive animated conversational graphical user interface with an avatar in the form of a human as shown in FIG. 3.

At step 210, the system waits for a user to position in front of one or more depth cameras. For example, 305 (FIG. 3) shows a user positioned in front of one or more depth cameras with the indication, “Medication Not Taken.”

At step 215, a determination is made if a hand and face are visible. If so, at step 220 the depth camera begins recording frames and the user is instructed to take a medication. If no hand and face are visible, the user returns to step 210.

At step 225, a determination is made if a back of a hand is visible while the face is occluded. If so, at step 230 medication compliance is detected. For example, 405 shows a user positioned in front of one or more depth cameras with the indication, “Medication Taken.” If the back of the hand is not visible and the face is not occluded, medication compliance is not detected.

FIG. 3 shows an exemplary specific, structured interactive animated conversational graphical user interface 300 with an avatar in the form of a human. 305 also shows a user positioned in front of one or more depth cameras.

According to various exemplary embodiments, a three-dimensional avatar in the form of a human as depicted in FIG. 3 functions to guide the user (such as user 305) through the data entry process in an effort to reduce user errors. This is achieved through the utilization of multiple cloud-based resources connected to the conversational interface system. For the provision of responses from the avatar to user inquiries, either Speech Synthesis Markup Language (SSML) or basic text files are read into the system and an audio file is produced in response. As such, the aspects of the avatar's response settings such as voice, pitch and speed are controlled to provide unique voice characteristics associated with the avatar during its response to user inquiries.

As illustrated in FIG. 3, the system waits for a user to position in front of one or more depth cameras. For example, 305 shows a user positioned in front of one or more depth cameras with the indication, “Medication Not Taken.” Subsequently, a determination is made if a hand and face are visible. If so, the depth camera begins recording frames and the user is instructed to take a medication.

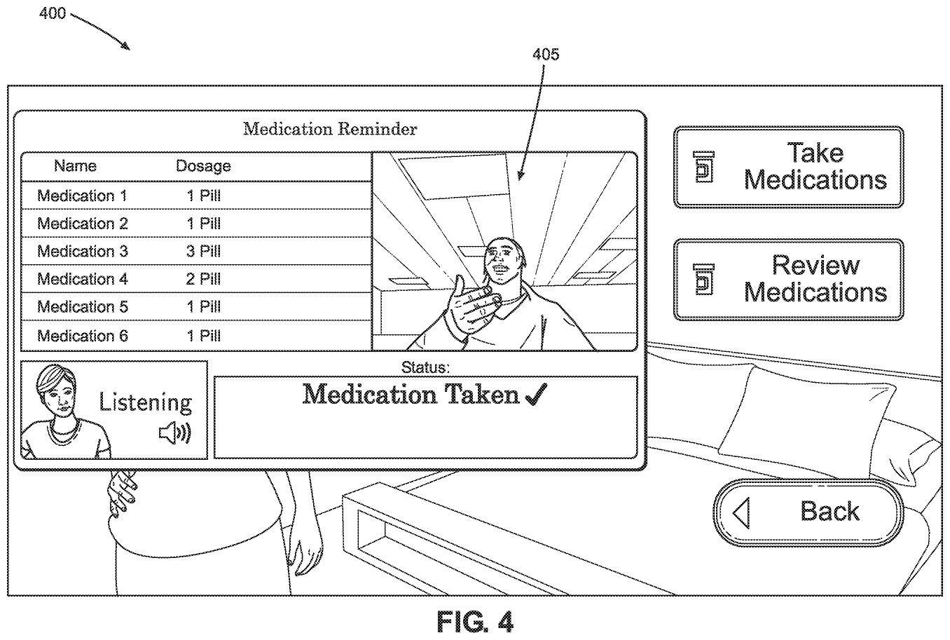

FIG. 4 shows another exemplary specific, structured interactive animated conversational graphical user interface 400 with an avatar in the form of a human. 405 also shows a user positioned in front of one or more depth cameras.

As illustrated in FIG. 4, the system makes a determination if a back of a hand is visible while the face is occluded. If so, medication compliance is detected. For example, 405 (FIG. 4) shows a user positioned in front of one or more depth cameras with the indication, “Medication Taken.”

While various embodiments have been described herein, it should be understood that they have been presented by way of example only, and not limitation. The descriptions are not intended to limit the scope of the technology to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the technology as defined by the appended claims and otherwise appreciated by one of ordinary skill in the art. The scope of the technology should, therefore, be determined not with reference to the description, but instead should be determined with reference to the appended claims along with their full scope of equivalents.

What is claimed is:

1. A computing device comprising a display screen, the computing device being configured to dynamically display a specific, structured interactive animated conversational graphical user interface paired with a prescribed functionality directly related to the interactive graphical user interface's structure.

2. The computing device of claim 1, further comprising a first computer vision model and a second computer vision model.

3. The computing device of claim 2, further comprising the first computer vision model configured to track a hand of a human.

4. The computing device of claim 3, further comprising the second computer vision model configured to track a face of a human.

5. The computing device of claim 4, further comprising the computing device programed with heuristic logic executed by a processor.

6. The computing device of claim 5, the heuristic logic including the first computer vision model determining if the hand is visible.

7. The computing device of claim 6, the heuristic logic including the second computer vision model determining if the face is visible.

8. The computing device of claim 7, the heuristic logic including the first computer vision model determining if a back of the hand is visible.

9. The computing device of claim 8, the heuristic logic including the second computer vision model determining if the face is occluded.

10. The computing device of claim 9, the heuristic logic inferring if (i) the hand is visible, (ii) the face is visible, (iii) the back of the hand is visible, and (iv) the face is occluded, then a medication has been taken by a human.

11. The computing device of claim 10, further comprising a computer vision model configured to track a throat of the human to detect a swallow by the human.

12. The computing device of claim 11, further comprising a computer vision model configured to detect a pill type.

13. The computing device of claim 12, being any form of a computing device, including a personal computer, laptop, tablet, or mobile device.

14. The computing device of claim 13, where upon initiation, a user is provided one or more options to select a desired method for data entry, including voice, type, touch or combinations thereof without having to switch back and forth.

15. The computing device of claim 14, further comprising user provided data being validated based on characteristics defined within the specific, structured interactive animated conversational graphical user interface.

16. The computing device of claim 15, further comprising user provided data being further validated against external data stored in a cloud-based database.

17. The computing device of claim 16, further comprising the specific, structured interactive animated conversational graphical user interface completing and updating a database entry.

18. The computing device of claim 17, further comprising the specific, structured interactive animated conversational graphical user interface converting text data to voice data for storage and for use in human conversation.

19. The computing device of claim 18, further comprising the specific, structured interactive animated conversational graphical user interface converting response data to audio files using cloud-based text-to-speech solutions capable of being integrated into a web browser based avatar.

20. The computing device of claim 19, further comprising the specific, structured interactive animated conversational graphical user interface including a virtual avatar in a form of a human for providing guidance and feedback to a user during utilization of the specific, structured interactive animated conversational graphical user interface.
